(19) United States
(12) Patent Application Publication
(10) Pub. No.: US 2013/0008454 A1
(43) Pub. Date: Jan. 10, 2013

(54) CHINSTRAP

(76) Inventor: Kathrin Suzanne Reams, Loveland, CO (US)

(21) Appl. No.: 13/542,601

(22) Filed: Jul. 5, 2012

Related U.S. Application Data
(60) Provisional application No. 61/505,499, filed on Jul. 7, 2011.

(51) Int. Cl. A01N 1/00; A61F 5/37

(52) U.S. Cl. 128/869; 27/25.1

(57) ABSTRACT

A chinstrap having a band portion with at least two head straps, one head strap fitting around a crown of a head of a person wearing the chinstrap, and one head strap fitting around a back of the head. The chinstrap has an adjustment mechanism to change a size of the band portion. The chinstrap also has a form-fitting non-slip chin pocket attached to the band portion. In an example, the chinstrap may be used to assist in holding the mouth of the person closed after death until a medication to freeze jaw muscles is injected following death during an embalming process so that an invasive mandibular septum stitch is not needed to secure the mouth closed.

### PRIORITY CLAIM

This application claims the benefit of U.S. Provisional Patent Application No. 61/505,499 filed on Jul. 7, 2011 for “Comfort Care Chinstrap” of Kathrin Suzanne Reams, incorporated by reference for all that is disclosed as though fully set forth herein.

### BACKGROUND

Failing to keep the patient's mouth closed can be distressing for family members or others who visit the patient. During hospice care, caretakers for a patient frequently receive requests from family and friends visiting the patient, for a way to keep the patient's mouth closed during the end stages of life and/or following death. If the patient dies with his or her mouth open, the jaw often has to be manipulated and an invasive procedure called mandibular septum stitch used during embalming in order to close the person's mouth after death, for example, for an open casket viewing.

After death caretakers may prop rolled up towels under the patient's chin, supporting the jaw, and behind the back of the head/neck tilting the head forward, but this does not work very well because if there is movement, the towels may fall away. The towels also clutter the patient's space and creates an unnatural looking anatomical position post mortem.

### BRIEF DESCRIPTION OF THE DRAWINGS

FIG. 1 is a perspective view of an example chinstrap.

FIGS. 2a-e are plan views of the example chinstrap shown in FIG. 1, wherein (a) is a front view, (b) is a rear view, (c) is one side view and the other side view is a mirror image, (d) is a top view, and (e) is a bottom view.

FIG. 3 is a perspective view of an example disposable chinstrap.

FIG. 4 is a perspective view showing additional features of an example chinstrap.

FIGS. 5a-b is an example sewing pattern for the chinstrap shown in FIG. 1.

FIG. 6 is an example sewing pattern for the disposable chinstrap shown in FIG. 3.

### DETAILED DESCRIPTION

A chinstrap is disclosed, as it may be used in an example as an end-of-life comfort care chinstrap preceding and/or immediately following death. After death, the chinstrap provides a gentle clamping tension and exerts a connecting force from the top of the head to below the jaw, in effect cinching the mouth closed. The personal chinstrap may be designed of soft stretchy fabric, such as cotton spandex, with soft loop and hook straps, with top strap adjustable with soft hook and loop. A disposable chinstrap may be designed of soft stretchy fabric, such as nylon mesh or elasticized non-woven material, with two straps, back and top, with top strap adjustable with soft hook, providing a snug fit as needed to secure a closed mouth post mortem and to accommodate different head sizes.

The comfort care chinstrap may be worn with a cup or pocket under the chin of a person, and straps or bands fitting over the top and back of the person's head. The chinstrap (or portion of the chinstrap, such as the pocket portion) may be constructed of a material that can be wiped clean. The disposable chinstrap does not need to have a fitted chin pocket. For example, the nylon mesh or elasticized non-woven material of the disposable chinstrap may conform snugly and secure placement around the person's chin.

The chinstrap may be used to assist in closure or partial closure of a dying person's mouth while sleeping and/or resting. The comfort care chinstrap may also assist in the closure of a deceased person's mouth until rigor mortis sets in (taking approximately 2-4 hours in the face, neck and jaw). This is especially beneficial if family and friends choose not to have the body removed by the mortuary right away, but instead are waiting for other family and/or friends to arrive, for viewing and to say their good-byes. The deceased person's dignity remains intact by normalizing the appearance with the mouth closed or partially closed. In another example, the chinstrap may be used by the mortuary until a medication can be injected, for example during the embalming process, which then freezes the jaw muscles and holds the mouth shut so that the chinstrap can be removed, providing a noninvasive alternative to the mandibular septum stitch, maintaining the dignity of a deceased body.

An example chinstrap may be produced in a variety of fabric colors and designs, accommodating different preferences of the patient and/or family members. Other customization, such as iron-on transfers may also be provided, so that patients and family can personalize the chinstrap for their loved ones. For example, if the dying person was an avid gardener, a rose tattoo may be ironed on. Or if the person was a truck driver all his life, a picture of a semi-truck may be ironed on. Messages of love and hope may also be written on the chinstrap using a permanent felt pen specifically designed for writing on fabric. People may also attach their own pins, for example, the American flag or graduation pin or anything of decoration and/or significance to them. In an example, the chin pocket may be interchangeable so that it can be washed and/or replaced with a different color or design pockets.

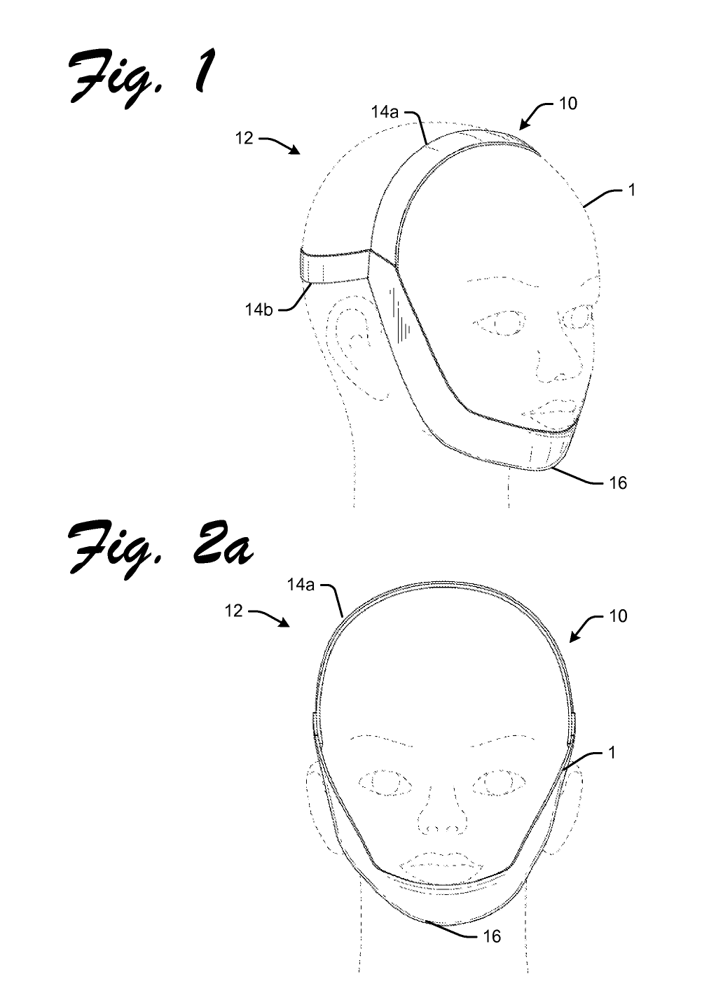

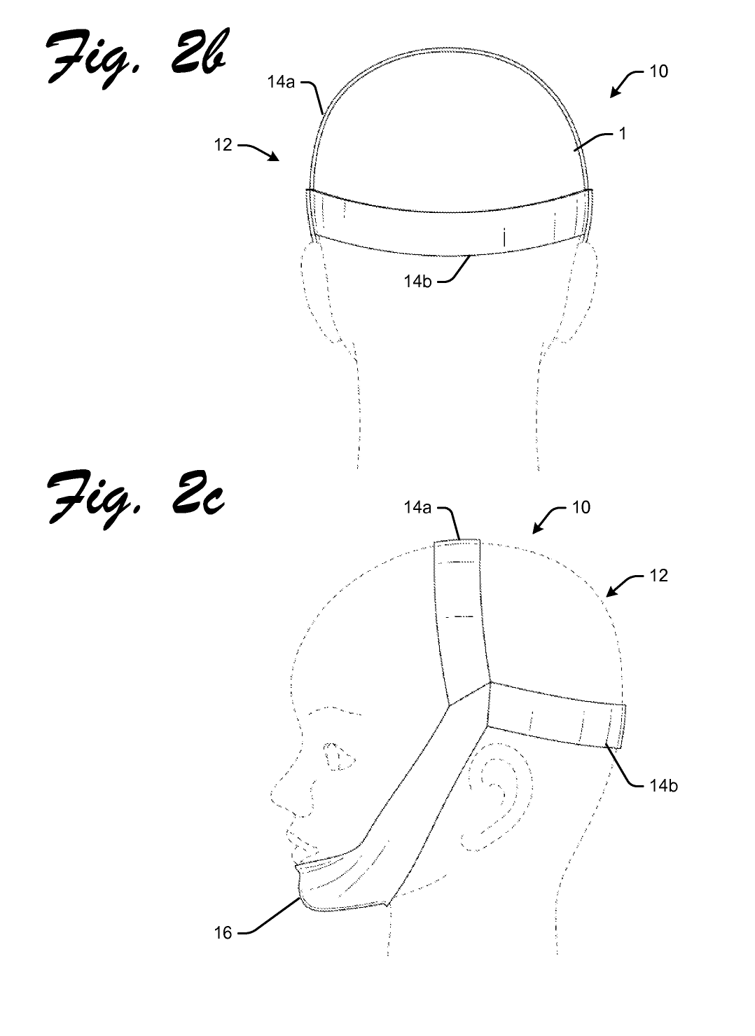

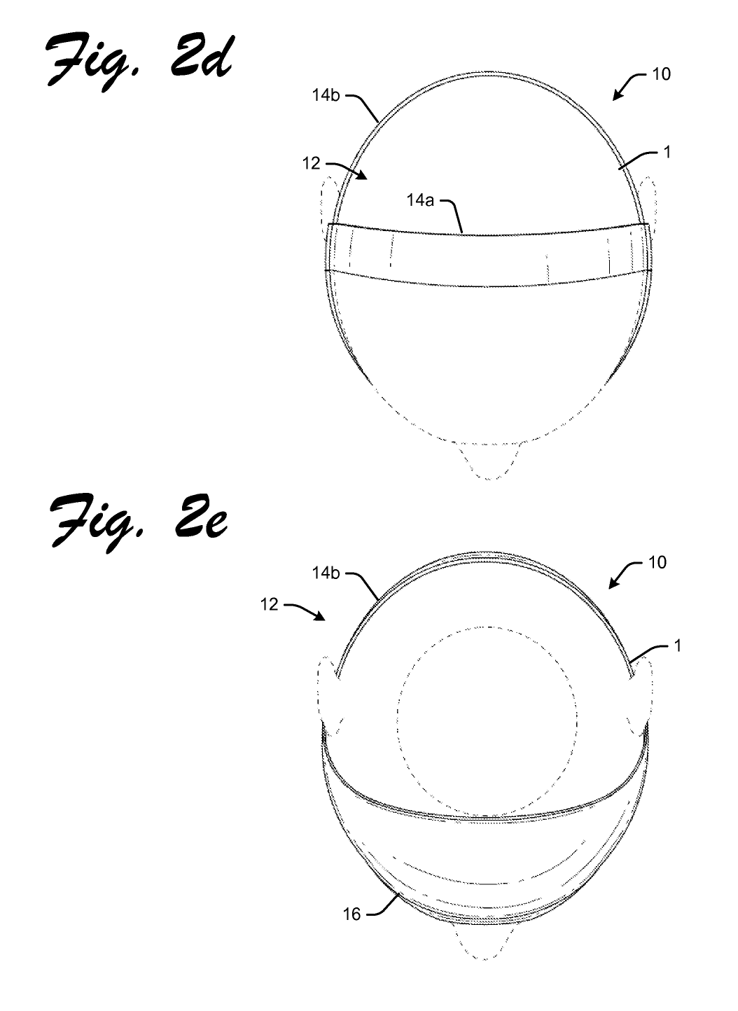

FIG. 1 is a perspective view of an example chinstrap 10. FIGS. 2a-e are plan views of the example chinstrap 10 shown in FIG. 1, wherein (a) is a front view, (b) is a rear view, (c) is one side view and the other side view is a mirror image, (d) is a top view, and (e) is a bottom view.

The example chinstrap 10 is shown including a band portion 12 with at least two head straps. One head strap 14a may be fitted around a crown of a head 1 of a person wearing the chinstrap. Another head strap 14b may be fitted around a back of the head 1. The chinstrap 10 also includes a form-fitting non-slip chin pocket 16 attached to the band portion 12.

The chin pocket 16 assists in the closure or partial closure of a mouth of the person to promote comfort, peace and dignity of the person. The chin pocket 16 may also assist in the closure or partial closure of a mouth of the person at least immediately after to death to maintain dignity of the deceased person. For example, the chin pocket 16 may assist in holding the mouth of the person closed after death until a medication to freeze jaw muscles is injected following death during an embalming process so that an invasive mandibular septum stitch is not needed to secure the mouth closed.

In an example, the band portion 12 of the chinstrap is narrower than the chin pocket 16. The chin pocket 16 may be made of a soft, stretchable cotton spandex fabric. In addition, the chin pocket may be made of a soft, stretchable plastic/elastic material that may be wiped clean as opposed to machine washed, for use by the mortuary. In addition, the chin pocket 16 may include a non-slip area on the portion to be worn under the person's chin. The band portion 12 may be made of a soft, stretchable hook and loop strapping.

It is noted that while wearing the chinstrap 10, a person can eat, drink, brush teeth, perform oral care, talk, etc., and the chinstrap does not interfere with any of these normal functions.

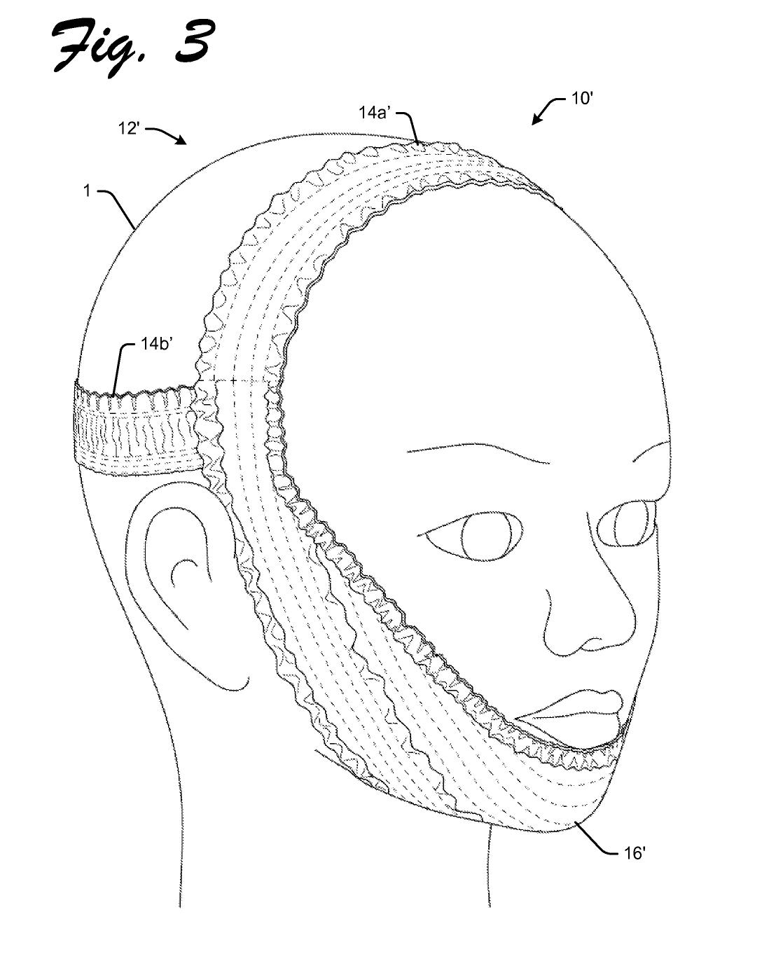

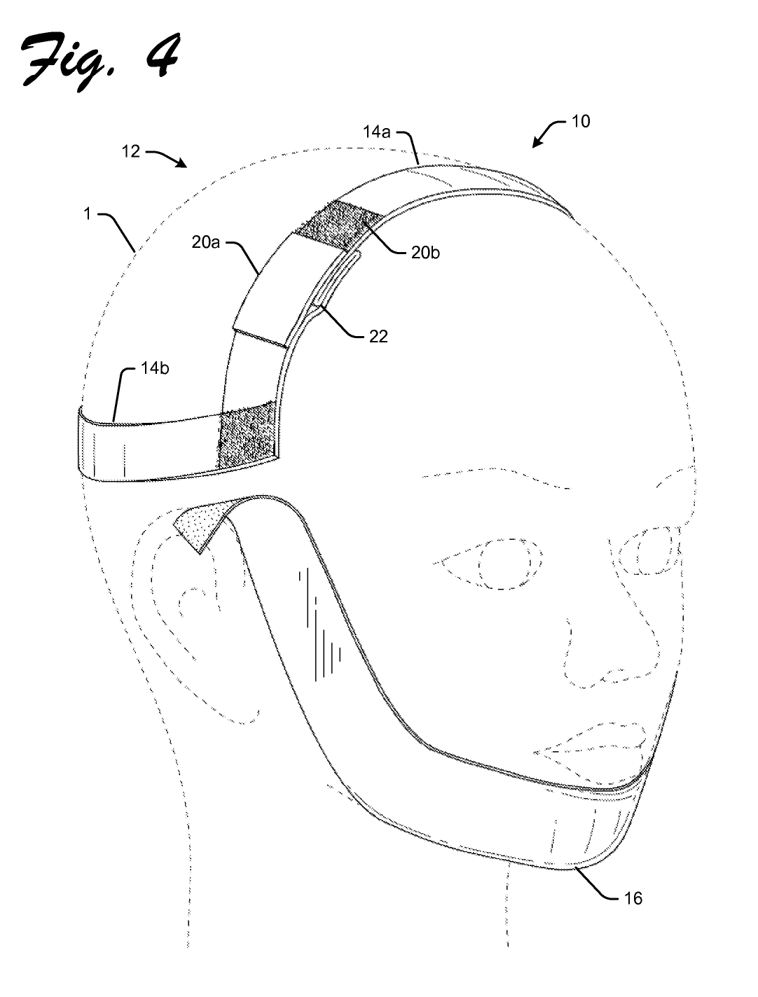

FIG. 3 is a perspective view of an example disposable chinstrap 10′. In this example, the chin pocket 16′ and band 12′ are made of a disposable material. In an example, the disposable chinstrap 10′ may be designed of soft stretchy fabric, such as nylon mesh or elasticized non-woven material. To reduce cost of the disposable chinstrap 10′, the material may be selected to provide a snug or tight fit without needing adjustment. Although not visible in FIG. 3, an adjustment mechanism may also be provided, as described in more detail below with reference to FIG. 4.

FIG. 4 is a perspective view showing additional features of an example chinstrap. The chinstrap 10 (or disposable chinstrap 10′) may include an adjustment mechanism 18 to change a size of the band portion 12, for example, if needed for a smaller persons head size or if the chinstrap needs to fit snugger post mortem. The adjustment mechanism 18 may include a hook and loop adjustment for fitting purposes, wherein the adjustment mechanism 18 includes overlapping hook and loop flaps or tabs. For example, hook tab 20a can be pulled over and attached to loop tab 20b to gather a portion 22 of the strap 14a and thus tighten the band portion 12 on the head 1. In an example, only one of the at least two head straps (e.g., strap 14a) is adjustable with the adjustment mechanism 18, and other of the at least two head straps (e.g., strap 14b) is a fixed size. Another adjustment mechanism for the disposable chinstrap may include only a hook tab and the hooks may “grab” the disposable material (similar to the hooks used to fasten an adult diaper).

Also as shown in FIG. 4, the chin pocket 16 can also be removable. In an example, hook and loop or other fastener (e.g., a button, not shown) may be provided. Accordingly, the chin pocket 16 may be interchangeable with another chin pocket, for example, to change color or design of the chin pocket, for cleaning, or other purpose.

Still other examples of the chinstrap are also contemplated. For example, the chinstrap 10 may also include side pockets for the insertion of ice packs or heat packs and enlarge the width at the jaw. The chinstrap 10 may also include side straps, e.g., using soft hook and loop, to secure oxygen tubing or a CPAP apparatus.

A pocket may be created with the label and may be used for the purpose of aromatherapy by tucking in a cotton ball moist with a few drops of essential oil.

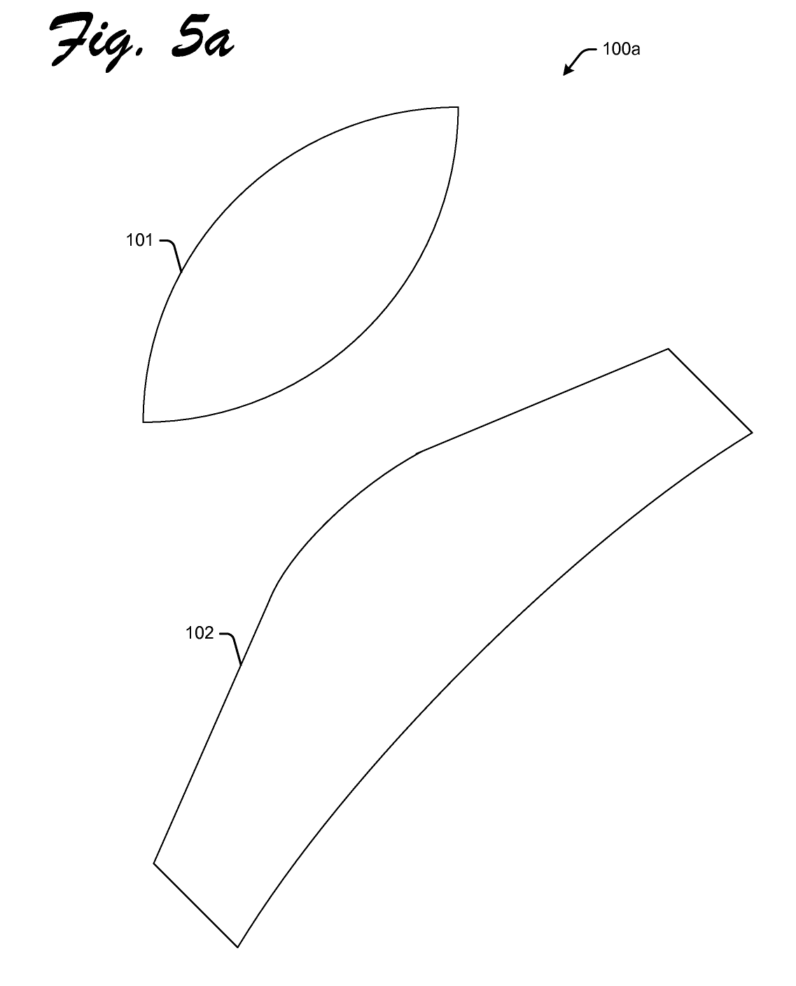

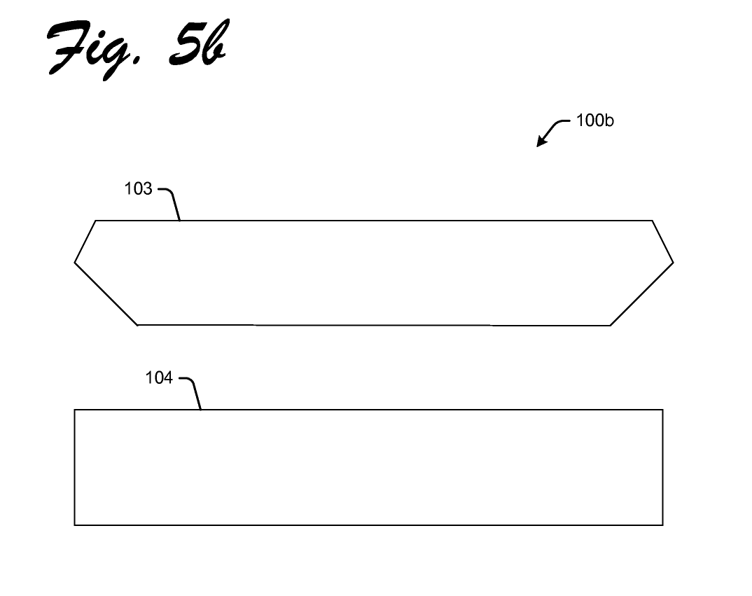

FIGS. 5a-b is an example sewing pattern 100a-b for the chinstrap shown in FIG. 1. The sewing pattern 100a-b includes a chin piece 101 (e.g., made of fabric), a body portion 102 (e.g., made of fabric), a back strap 103 (e.g., made of elastic loop), and a front strap 104 (e.g., made of elastic loop). In an example, the chinstraps disclosed herein may be manufactured as follows.

Cutting Instructions:

1. Cut Sheet

1a. Cotton Spandex:
Cut 2 BODY—use pattern
Cut 2 CHIN PIECE—use pattern

1b. Soft Elastic Loop:
Cut 1 BACKSTRAP—use pattern
Cut 1 FRONT STRAP—10″/use pattern, mark center line

1c. Soft Hook
Cut 1 @ 2″
1 label

Example sewing instructions include:
1. Sew CHIN PIECE to BODY matching center notch, using ⅜″ seam allowance.
2. Lay BODY, face sides together. Start at end and sew around the

CHIN PIECE, opening the seam allowance and continue to other end, using ⅜″ seam allowance.
3. Open the BODY and zigzag along one side of the seam line in #2. Both seam allowances are being stitched down. (This supports the seam in Step #2, in the event the fabric is stretched to the point that the threads pop.)
4. Open seam allowances on CHIN PIECE and zigzag across, making sure both seam allowances lay open and flat. Backstitch both ends.
5. Open BODY, face up. Lay FRONT STRAP face down on zigzag side of center line, then lay the BACK STRAP face up on top, fold other side of BODY around and stitch closed using ⅜″ seam allowance.
6. Repeat #10 on opposite side.
7. Serge the unfinished edge of BODY and CHIN.
8. Fold BACK STRAP away from BODY and stitch on serge line on BODY. (This holds the BACK STRAP in the correct position when worn.)
9. Attach HOOK at center line on FRONT STRAP and zigzag along other end of HOOK, inserting LABEL securing both pieces. LABEL is going one direction (e.g., left) and the HOOK is going the other direction (e.g., right).

The chinstrap 10 may be machine washable, using cold water, gentle cycle, hang to dry. Or the chinstrap may be disposable. The chinstrap 10 may also be available in a variety of colors and designs and sold in a variety of different sizes. The chinstrap 10 may also include customizing options, e.g., by attaching iron-on decals or pins to customize for individuals. Also available may be fabric pens for writing messages of love, hope, faith, encouragement, etc.

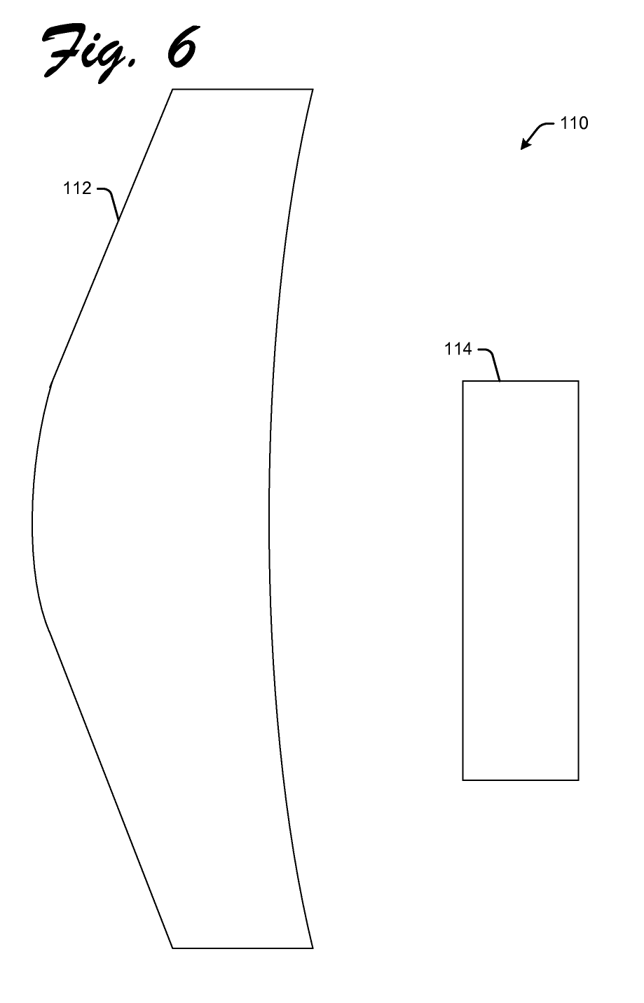

FIG. 6 is an example sewing pattern for the disposable chinstrap shown in FIG. 3. The sewing pattern 110 includes a body portion 112 (e.g., made of disposable material), and a back strap 114 (e.g., made of disposable material).

Example sewing instructions include:
1. Label is printed on HOOK TAB.
2. Place HOOK TAB on long end of FRONT/TOP.
3. Sew ends of FRONT/TOP together.
4. Sew BACK STRAP at notches on FRONT/TOP. Short edge is towards the curve on FRONT/TOP.

The chinstrap 10 may be worn by a person and helps promote comfort, peace, and restores dignity of the person wearing the chinstrap. Comfort may be provided to the person wearing the chinstrap by way of supporting weak jaw muscles and providing the sensation of a “facial hug.” Peace may be provided by way of providing choice in the dying process and helping caregivers feel that they have been able to help and achieve comfort, dignity and special care for their loved one. Using the chinstrap is a tangible way for family/friends/care providers, to do something with important benefits for the person dying. Dignity may be provided by way of normalizing appearance, maintaining a peaceful look, pre- and post-mortem. When the comfort care chinstrap is worn after death occurs, it enables the person's mouth to be closed and/or partially closed as rigor mortis sets in, bringing comfort and peace to grieving family and friends.

Other possible uses for the chinstrap 10 includes use as a post death chinstrap to keep the person's mouth closed or partially closed until rigor mortis sets in (2-4 hours) and/or a medication is administered by a mortician to “freeze” the jaw muscles. The chinstrap 10 may also be used to aid in prevention or reduction of snoring. The chinstrap 10 may also be used to aid in preventing clenching and or grinding of teeth while sleeping. The chinstrap 10 may also be used to aid in relief for tempromandibular joint (TMJ) sufferers by holding ice and/or heat packets in place (e.g., in a pocket in the chinstrap, not shown in the Figures) and relaxing the person's jaw.

The chinstrap 10 may also be used to use for post-op patients that are heavily medicated after surgery and sleeping on their backs, to keep the person's mouth from opening. The chinstrap 10 may also be used during post-op facial surgery or plastic surgery, to aid in securing facial bandages and prevent over stretching of the jaw muscles. Some surgeries require that the patient refrain from smiling or laughing afterwards for 24 to 72 hours. The chinstrap 10 may also be used for post-op dental surgery, to help the jaw relax and hold ice packs in place. The chinstrap 10 may also be used for post-op eye surgery, to help secure bandages in a desired position.

The chinstrap 10 may also be used to aid in securing oxygen tubing. This may be especially useful for patients who are developing sores behind their ears from the oxygen tubing.

The chinstrap 10 may also be used to help secure continuous positive airway pressure device for sleep apnea (CPAP). The chinstrap 10 may also be used for people who regularly sleep with their mouths open and would prefer not to.

It is noted that the exemplary embodiments shown and described are provided for purposes of illustration and are not intended to be limiting. Still other embodiments are also contemplated.

1. A chinstrap, comprising:
a band portion with at least two head straps, one head strap fitting around a crown of a head of a person wearing the chinstrap, and one head strap fitting around a back of the head;
an adjustment mechanism to change a size of the band portion; and
a form-fitting non-slip chin pocket attached to the band portion.

2. The chinstrap of claim 1, wherein the chin pocket assists in the closure or partial closure of a mouth of the person to promote comfort, peace and dignity of the person.

3. The chinstrap of claim 1, wherein the chin pocket assists in the closure or partial closure of a mouth of the person immediately after to death to promote comfort, peace and dignity following death of the person.

4. The chinstrap of claim 1, wherein the chin pocket assists in holding the mouth of the person closed after death until a medication to freeze jaw muscles is injected following death during an embalming process so that an invasive mandibular septum stitch is not needed to secure the mouth closed.

5. The chinstrap of claim 1, wherein the band portion is narrow and the chin pocket is wide, relative to one another.

6. The chinstrap of claim 1, wherein the band portion has a non-slip area on the portion to be worn under the person's chin.

7. The chinstrap of claim 1, wherein the chin pocket is made of a soft, stretchable fabric.

8. The chinstrap of claim 1, wherein the band portion is made of a soft, stretchable hook and loop strapping.

9. The chinstrap of claim 1, wherein the chin pocket is removable.

10. The chinstrap of claim 8, wherein the chin pocket is interchangeable with another chin pocket to change color or design of the chin pocket.

11. The chinstrap of claim 1, wherein the chin pocket and band portion is made of a disposable material.

12. The chinstrap of claim 1, wherein the adjustment mechanism includes overlapping hook and loop flaps.

13. The chinstrap of claim 1, wherein only one of the at least two head straps is adjustable with the adjustment mechanism, and other of the at least two head straps is a fixed size.

14. A chinstrap to assist in closure or partial closure of a mouth of a person before and after death of the person, the chinstrap promoting comfort, peace and dignity following death of the person, the chinstrap comprising:
a soft, stretchable band means for fitting around a crown of a head of a person wearing the chinstrap, and for fitting around a back of the head;
an adjustment means for changing a size of the band portion; and
a pocket means for supporting a chin of the person, the pocket means attached to the band portion.

15. The chinstrap of claim 14, wherein the pocket means is for holding the mouth of the person closed after death until a medication to freeze jaw muscles is injected following death during an embalming process so that an invasive mandibular septum stitch is not needed to secure the mouth closed.

16. The chinstrap of claim 14, wherein the band means is narrower on the head straps and wider at a chin portion, and the band has a non-slip area on the portion to be worn under the person's chin.

17. The chinstrap of claim 16, wherein the pocket means is interchangeable with another chin pocket to change color or design of the chin pocket.

18. The chinstrap of claim 14, wherein the pocket means and band means are disposable.

19. A method to assist in closure or partial closure of a mouth of a person immediately after death of the person for promoting comfort, peace and dignity of the deceased person, the method comprising:
fitting a first band around a crown of a head of a person, and fitting a second band around a back of the head, the first and second bands connected to a chin support;
adjusting at least one of the first and second bands to fit on the head; and
supporting a chin of the person with the chin support.

20. The method of claim 19, further comprising holding the mouth of the person closed after death until a medication to freeze jaw muscles is injected following death during an embalming process so that an invasive mandibular septum stitch is not needed to secure the mouth closed.
